(19) United States
(12) Patent Application Publication
(10) Pub. No.: US 2009/0159229 A1
(43) Pub. Date: Jun. 25, 2009

(54) Functional Composition, and Method for Improvement in Detachability of Wet Paper Using the Same

(76) Inventors: Hiroshi Sekiya, Tokyo (JP); Hiraku Sawada, Shizuoka (JP); Kanae Ichimura, Shizuoka (JP); Atsutoshi Kimura, Shizuoka (JP); Mineo Watanabe, Shizuoka (JP); Masato Takahashi, Shizuoka (JP); Masahiro Hamasaki, Shizuoka (JP); Seiichiro Usui, Shizuoka (JP)

Correspondence Address: FLYNN THIEL BOUTELL & TANIS, P.C., 2026 RAMBLING ROAD, KALAMAZOO, MI, 49008-1631, US

(21) Appl. No.: 12/087,329

(22) PCT Filed: Dec. 19, 2006
(86) PCT No.: PCT/JP2006/325257

§ 371 Date: Nov. 13, 2008

(30) Foreign Application Priority Data
Dec. 28, 2005 (JP) 2005-380491
Jul. 3, 2006 (JP) 2006-183892

(51) Int. Cl. D21F 11/00; C07C 43/04; C08G 65/00

(52) U.S. Cl. 162/210; 568/679; 525/50

(57) ABSTRACT

[Problems] To provide a functional composition enable a stale paper production by being applied onto a press roll, and a method for improvement of detachability using the same.
[Solutions] The present invention relates to a functional composition used by being applied onto a press roll for dehydration of a wet paper, comprising a compound expressed by the following general formula (1):
STR

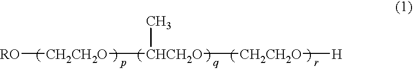

wherein R represents an organic group which may have a substituent or a hydrogen atom, p and r each independently represent an integer of 0 to 228, and q represents an integer of 0 to 69, provided that p, q and r do not represent 0 simultaneously.

### TECHNICAL FIELD

The present invention relates to a functional composition, and a method for improvement in detachability of a wet paper using the same.

### BACKGROUND ART

In a paper making process to produce paper, generally, there are a wire part that a liquid in which pulp is dispersed in water is mounted on a net (wire) for paper making, excess water is fallen naturally to be a wet paper, a press part that the wet paper is passed through a pair of press rolls and pressed by the press rolls via felt for water in the wet paper to be transferred, thereby to dehydrate the wet paper, a dryer part that the wet paper passed through the press part is contacted with a heated drum for drying into paper, and a reel part that the paper is wound around a bar called a spool.

However, in the conventional press part, since a press roll is rotated at a high speed, after a wet paper is passed through a point that it is pressed by press rolls (hereinafter referred to as “pressing point”), there is a case where it is not detached from the press roll and the press roll rotates while attaching it thereon.

Specifically as shown in FIG. 5, a wet paper is pressed at a pressing point P, and it is detached from the surface of press roll and transferred.

When a wet paper stays being not detached from the surface of press roll, excess tension is loaded on the wet paper, paper habit becomes bad, and a situation of paper breakage occurs.

On the other hand, in the above-described press part, generally, on a press roll of the side contacting a wet paper, there is disposed a doctor blade for removing a foreign material attached on the surface of the press roll so as to be contacted.

However, since the press roll rotates at a high speed, there is a case that a contact part of the doctor blade with the press roll is abraded by friction of the doctor blade and press roll to generate a gap between the doctor blade and press roll.

This is noticeably observed particularly when the doctor blade is made of carbon.

In this case, because a foreign material on the surface of press roll is not sufficiently removed, a wet paper is pressed by press rolls together with a foreign material, which causes defects that a wet paper is holed or the like.

From these reasons, there is a problem that no stable production can be performed in the conventional paper making process.

To these problems, a method that chemicals are applied onto press rolls has been proposed.

For example, during paper making processes, disclosed is a method for improvement in detachability of a wet paper from a granite roll where in a press part of dehydration part of a wet paper, detachment of a wet paper from a granite roll is markedly improved by pouring a granite roll-stainproof agent composed of a predetermined component as an effective component onto the surface of granite roll or felt, leading to improvement of productivity (see Patent document 1).

Further, disclosed is a method for preventing abrasion of a doctor blade where in a paper making process that attachments of the press roll are removed by pressure-contact of a doctor blade with the press roll as well as a wet paper containing a lot of water is sandwiched by a pair of press rolls to dehydrate the wet paper, a fluorinated organic compound with a predetermined structure is attached on the press roll (see Patent document 2).

Patent document 1: Japanese Unexamined Patent Publication No. Hei 11-217787 (1999)
Patent document 2: Japanese Unexamined Patent Publication No. 2005-273026

### DISCLOSURE OF THE INVENTION

However, according to the method described in Patent document 1, although detachability of a wet paper to press rolls is improved, it cannot be said to be sufficient.

Suppressing for abrasion of a doctor blade is also insufficient.

On the other hand, according to the method described in Patent document 2, although abrasion of a doctor blade can be relatively suppressed, detachability of a wet paper to press rolls is insufficient.

Therefore, the methods described in Patent documents 1 and 2 do not satisfy both detachability of a wet paper to press rolls and lubricity of a doctor blade.

Namely, since the above-described detachability cannot be maintained by the methods described in Patent documents 1 and 2, breakage of paper, a doctor blade wears because the above-described lubricity is insufficient, and a foreign material tends not to be removed sufficiently.

Hence, a stable production is difficult by the methods described in Patent documents 1 and 2.

The present invention was achieved in view of the problems resulting from the conventional arts, it is an object to provide a functional composition enable a stable paper production by being applied onto a press roll, and a method for improvement in detachability of a wet paper using the composition.

A functional composition of the present invention to solve the above-described problems is used by being applied onto a press roll for dehydration of a wet paper, and characterized by comprising the compound expressed by the following general formula (1):

### [STR1]

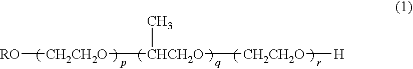

wherein R represents an organic group which may have a substituent or a hydrogen atom, p and r each independently represent an integer of 0 to 228, and q represents an integer of 0 to 69, provided that p, q and r do not represent 0 simultaneously.

When this functional composition is applied onto a press roll for dehydration of a wet paper, detachability of the wet paper to the press roll is improved.

From this, even when a press roll rotates at a high speed, detachment of a wet paper is maintained and breakage of paper is suppressed.

In addition thereto, since lubricity of the surface of a press roll is improved, abrasion due to friction of a doctor blade with the press roll is suppressed.

Therefore, it is suppressed that a contact part of the doctor blade with the press roll is abraded by friction of the doctor blade and press roll to generate a gap between the doctor blade and press roll.

Further, since abrasion of a doctor blade is suppressed, a foreign material on the surface of press roll is removed sufficiently, and it is possible to suppress generation of defect that a wet paper is holed or the like.

Furthermore, since abrasion of a doctor blade is suppressed, it is possible to decrease the frequency of exchange of doctor blades.

Hence, according to the functional composition of the present invention, by applying it on a press roll, abrasion of a doctor blade can be suppressed as well as detachability of a wet paper to the press roll can be improved.

Therefore, a stable production becomes possible.

As described above, reasons that the foregoing effect is obtained by applying the functional composition of this case onto a press roll are not certain, but it is assumed that surface tension of water is lowered, thus a membrane that adhesion force of a wet paper was lowered is formed on the surface of a press roll.

Additionally, the factor is not limited thereto.

It is preferable that the above-described functional composition further comprises a water-soluble polymer.

In this case, when the functional composition is applied onto a press roll, a membrane of the water-soluble polymer is formed on the surface of a press roll together with the compound expressed by the general formula (1), thus it is possible to maintain high detachability of a wet paper to the press roll even in the case of using the press roll for a long period of time.

In the above-described functional composition, it is preferable that R is a hydrogen atom, p and r each independently are an integer of 27 to 228, and q is an integer of 25 to 69.

By applying the functional composition of this case onto a press roll, abrasion of a doctor blade can be further suppressed as well as detachability of a wet paper to the press roll can be further improved.

Therefore, a more stable production becomes possible.

In the above-described functional composition, it is preferable that R is a hydrocarbon group having carbon numbers of 10 to 16 which may have a substituent, the sum of p and r is an integer of 6 to 30, and q is an integer of 0 to 2.

By applying the functional composition of this case onto a press roll, abrasion of a doctor blade can be further suppressed as well as detachability of a wet paper to the press roll can be further improved.

Therefore, a more stable production becomes possible.

In the above-described functional composition, it is preferable that the content ratio of ethylene oxide group expressed by the following general formula (2) to the molecular weight of the compound expressed by the general formula (1) is 30% by mass or more.

### [STR2]

CH₂CH₂O (2)

In this case, since friction between a doctor blade and a press roll is further reduced, abrasion of a doctor blade is further suppressed.

In the above-described functional composition, it is preferable that the mixing ratio of the compound expressed by the general formula (1) and the water-soluble polymer is 1:0.1 to 1:10 in mass ratio.

In this case, when the mixing ratio of the compound expressed by the general formula (1) and the water-soluble polymer in mass ratio is in the above-described range, a further uniform membrane is formed.

In the above-described functional composition, it is preferable that the press roll is equipped with a doctor blade for removing a foreign material attached onto the surface.

It is preferable that the above-described functional composition is a remover to improve detachability of a wet paper from the press roll.

Further, it is preferable that the above-described functional composition is a friction reducing agent to improve lubricity between the doctor blade and press roll.

In these cases, by applying the functional composition onto a press roll, abrasion of a doctor blade can be surely suppressed as well as detachability of a wet paper to the press roll can be surely improved.

Therefore, a more stable production becomes possible.

The method for improvement in detachability of a wet paper of the present invention is characterized by using the foregoing functional composition.

According to the present invention, since the foregoing functional composition is used, it is possible to improve detachability of a wet paper by applying the functional composition onto a press roll contacting a wet paper.

### EFFECT OF THE INVENTION

According to the present invention, it is possible to provide a functional composition enable a stable paper production because by applying it onto a press roll, abrasion of a doctor blade can be suppressed as well as detachability of a wet paper to a press roll can be improved, and a method for improvement of detachability using it.

### BEST MODE FOR CARRYING OUT THE INVENTION

Hereinafter, preferable embodiments of the present invention will be described in detail with reference to the drawings, if required.

Additionally, in the drawings, the same element is denoted by the same symbol and number, and repeated explanations are omitted.

Further, positional relations such as left and right, and up and down are based on the positional relations shown in the drawings unless otherwise specified.

Furthermore, dimensional ratios in the drawings are not limited to the ratios illustrated therein.

The functional composition of the present invention comprises the compound expressed by the following general formula (1):

### [STR3]

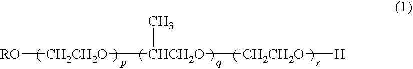

Herein, in the above general formula (1), R represents an organic group which may have a substituent or a hydrogen atom, p and r each independently represent an integer of 0 to 228, and q represents an integer of 0 to 69, provided that p, q and r do not represent 0 simultaneously.

As the above-described organic group, it is not particularly limited as long as it is a group containing a carbon atom, for example, there are listed a linear, branched or circular hydrocarbon group, and an aryl group such as a phenyl group and a naphthyl group.

Further, as the above-described substituent, there are listed an aryl group such as a phenyl group and a naphthyl group, a halogen group, an amino group, an alkylamino group, a carbonyl group, an ester group, a sulfonyl group, a nitro group, and an alkylene oxide group.

Additionally, the compound expressed by the following general formula (1) may have these substituents alone or a plurality thereof alone, or two kinds or more.

When the above-described functional composition is applied onto a press roll for dehydration of a wet paper, detachability of a wet paper to the press roll is improved.

From this, even when a press roll rotates at a high speed, detachment of a wet paper is maintained and breakage of paper is suppressed.

In addition thereto, since lubricity of the surface of a press roll is improved, abrasion due to friction of a doctor blade with the press roll is suppressed.

Therefore, it is suppressed that a contact part of the doctor blade with the press roll is abraded by friction of the doctor blade and press roll to generate a gap between the doctor blade and press roll.

Hence, by applying the functional composition of this case onto a press roll, abrasion of a doctor blade can be suppressed as well as detachability of a wet paper to the press roll can be improved.

Therefore, a stable production becomes possible.

In a compound expressed by the above-described general formula (1), when R is a hydrogen atom, the compound as expressed by the following general formula (3) is preferably, being a propylene oxide group as a center, a polyoxyethylene polyoxypropylene block polymer having ethylene oxide groups on the left and right.

### [STR4]

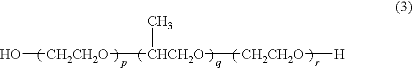

Herein, values of p, q and r in the general formula (3) are preferably in the following range from the viewpoint of lubricity.

Namely, p is preferably an integer of 27 to 228, and more preferably an integer of 38 to 199.

q is preferably an integer of 25 to 69, and more preferably an integer of 34 to 60.

r is preferably an integer of 27 to 228, and more preferably an integer of 38 to 199.

And, in addition thereto, it is more preferably that the values of p and r are the same.

Additionally, in a compound expressed by the general formula (1), the functional composition of the present invention may contain 2 kinds or more of the compounds that values of p, q and r are different.

In the case where R is a hydrogen atom, by applying the functional composition onto a press roll, abrasion of a doctor blade can be further suppressed as well as detachability of a wet paper to the press roll can be further improved.

Therefore, a more stable production becomes possible

Additionally, an average molecular weight of the propylene oxide group expressed by the following general formula (4) in the above-described polyoxyethylene polyoxypropylene block polymer is preferably 1300 or more from the viewpoint of attachability of the functional composition onto a press roll, and more preferably 1500 or more.

### [STR5]

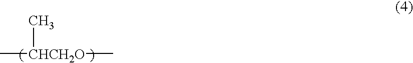

It is preferable that the above-described functional composition further comprises a water-soluble polymer.

In this case, when the functional composition is applied onto a press roll, a membrane of the water-soluble polymer is formed on the surface of a press roll together with the compound expressed by the general formula (3), thus it is possible to maintain high detachability of a wet paper to the press roll even in the case of using the press roll for a long period of time.

The above-described water-soluble polymer is preferably cationic, amphoteric or nonionic, and more preferably cationic or amphoteric.

These water-soluble polymers may be used alone, or in mixture of 2 kinds or more thereof.

Additionally, the amphoteric is obtained by polymerizing a cationic monomer with an anionic monomer.

In this case, there is an advantage that detachability of a wet paper is further improved.

As the above-described cationic water-soluble polymer, there are listed polydiallyldimethylammonium, poly(diallyldimethylammonium chloride), dicyandiamide-formamide condensate, epichlorohydrin-dimethylamine condensate and the like.

Further, the cationic water-soluble polymer may be a polymer that a halogenated amine derivative having a polymerizable functional group as a cationic monomer and a monomer having an ethylenic double bond as a nonionic monomer are polymerized.

As the above-described halogenated amine derivative having a polymerizable functional group, salt of (meth)acrylic acid with 2-(N,N-dimethylamino)ethylbenzene chloride, salt of (meth)acrylic acid with 2-(N,N-dimethylamino)ethyl chloride and the like are listed.

Further, as the above-described monomer having an ethylenic double bond, there are listed ethylene glycol mono(meth)acrylate, ethylene glycol di(meth)acrylate, diethylene glycol mono(meth)acrylate, diethylene glycol di(meth)acrylate, triethylene glycol mono(meth)acrylate, triethylene glycol di(meth)acrylate, propylene glycol mono(meth)acrylate and the like.

These monomers may be used alone, or in mixture of 2 kinds or more thereof.

As the above-described amphoteric water-soluble polymer, there is listed a polymer that a halogenated amine derivative having a polymerizable functional group as a cationic monomer and a carboxylic acid derivative having a polymerizable functional group as an anionic monomer are polymerized.

Additionally, in polymerizing the cationic monomer and the anionic monomer, the foregoing nonionic monomer may be polymerized at the same time.

As the above-described halogenated amine derivative having a polymerizable functional group, the same one as the foregoing one is used.

Further, as the above-described carboxylic acid derivative having a polymerizable functional group, (meth)acrylic acid and the like are listed.

These monomers may be used alone, or in mixture of 2 kinds or more thereof.

In the compound expressed by the foregoing general formula (1), when R is an organic group which may have a substituent, the organic group is preferably a hydrocarbon group having carbon numbers of 10 to 16, and more preferably a hydrocarbon group having carbon numbers of 12 to 15.

Additionally, it is preferable that the above-described hydrocarbon group does not have a substituent.

When carbon numbers are less than 10, surface tension becomes high compared with the case where the carbon numbers is in the above-described range, there is a tendency that membrane is not formed sufficiently, whereas when carbon numbers exceed 16, there is a tendency that the compound is not uniformly attached onto the surface of a press roll due to difficulty of uniform dispersion in water.

Additionally, in a compound expressed by the general formula (1), the functional composition of the present invention may contain 2 kinds or more of the compounds that carbon numbers are different.

In this case, the sum of p and r is preferably an integer of 6 to 30 from the viewpoint of compatibility with water, more preferably an integer of 6 to 20, further preferably an integer of 8 to 20, and further more preferably an integer of 9 to 16.

Further, q is preferably an integer of 0 to 2, and more preferably 0.

Namely, when q is 0, it is the compound expressed by the following general formula (5):

### [STR6]

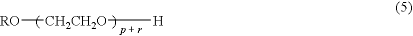

Among these, the compound expressed by the above-described general formula (1) is further preferably polyoxyethylene lauryl ether, polyoxyethylene tridodecyl ether, polyoxyethylene myristyl ether, and polyoxyethylene pentadecyl ether.

In the compound expressed by the above-described general formula (1), when the organic group is a hydrocarbon group, the functional composition of the present invention is used in coexistence with a water-soluble polymer.

Additionally, the water-soluble polymer herein is the same as the foregoing water water-soluble polymer.

In this case, when the above-described functional composition is applied onto a press roll, a membrane of the water-soluble polymer is formed on the surface of a press roll together with the compound expressed by the general formula (5), thus it is possible to maintain high detachability of a wet paper to a press roll even in the case of using the press roll for a long period of time.

In the case where R is an organic group which may have a substituent, by applying the functional composition on a press roll, abrasion of a doctor blade can be further suppressed as well as detachability of a wet paper to the press roll can be further improved.

Therefore, a more stable production becomes possible.

In the above-described functional composition, it is preferable that the content ratio of ethylene oxide group expressed by the following general formula (2) to the molecular weight of the compound expressed by the general formula (1) is 30% by mass or more, and 90% by mass or less.

### [STR7]

CH₂CH₂O (2)

When the content ratio of ethylene oxide group is less than 30% by mass, membrane strength tends to be insufficient compared with the case where the content ratio of ethylene oxide group is in the above-described range, when the content ratio of ethylene oxide group exceeds 90% by mass, the membrane tends not to be sufficiently attached onto a press roll compared with the case where the content ratio of ethylene oxide group is in the above-described range.

In the above-described functional composition, it is preferable that the mixing ratio of the compound expressed by the general formula (1) and the water-soluble polymer is 1:0.1 to 1:10 in mass ratio, and 1:0.25 to 1:4 is more preferable.

When the mass ratio of the water-soluble polymer to the functional composition 1 is less than 0.1, there is a tendency that membrane cannot be maintained sufficiently compared with the case where the mass ratio is in the above-described range, when the mass ratio of the water-soluble polymer to the functional composition 1 exceeds 10, there is a tendency that detachability is lowered due to an increase in tackiness compared with the case where the mass ratio is in the above-described range.

In the functional composition of the present invention, it is preferable that surface tension of the compound expressed by the general formula (1) is 25 to 40 mN/m.

In this case, it is possible to apply the functional composition onto the surface of a press roll further uniformly.

The functional composition of the present invention may contain, other than the compound expressed by the above-described general formula (1) and the water-soluble polymer, additives such as a chelating agent, pH adjusting agent, antiseptic, disperser, viscosity adjusting agent, and solid lubricant.

Next, a dehydration process of a wet paper using the foregoing functional composition will be explained.

The above-described functional composition is used in a paper making machine equipped with felt, a pair of press rolls for pressing a wet paper via the felt, and a doctor blade contacting a press roll of the side contacting a wet paper.

FIG. 1 is an explanatory diagram for explaining one example of constitution of a press part in a paper making machine that the functional composition of the present invention is used.

As shown in FIG. 1, a press part 100 includes a first press part A and a second press part B, dehydration is carried out at two places in the first press part A and one place in the second press part B.

In the first press part A, a press roll 10 as a center (hereinafter referred to as “center roll”) is equipped, a first press roll 11 and a second press roll 12 are provided so as to be able to press a wet paper at pressing points P1 and P2 on the surface of the center roll.

In other word, it is constituted in such a manner that the center roll 10 and the first press roll 11 can press a wet paper at the pressing point P1, and the center roll 10 and the second press roll 12 can press a wet paper at the pressing point P2.

On the other hand, in the second press part B, an upper press roll 15 (hereinafter referred to as “top roll”) is equipped, a third press roll 13 is provided so as to be able to press a wet paper at a press point P3 on the surface of the top roll 15.

In other word, it is constituted in such a manner that the top roll 15 and the third press roll 13 can press a wet paper at the pressing point P3.

A wet paper 30 is disposed so that it passes through between the center roll 10 and the first press roll 11, and through between the center roll 10 and the second press roll 12 in the first press part A, then passes through between the top roll 15 and the third roll 13 in the second press part B.

Further, when the wet paper 30 is pressed each at pressing points P1, P2 and P3, the wet paper 30 is set to be pressed via felts 21, 22 and 23 between the press rolls.

Namely, the felt 21 is disposed between the center roll 10 and the first press roll 11, contacting the first press roll 11.

Further, the felt 22 is disposed between the center roll 10 and the second press roll 12, contacting the second press roll 12.

Further, the felt 23 is disposed between the top roll 15 and the third press roll 13, contacting the third press roll 13.

Therefore, when the wet paper 30 passes through the first press part A, it contacts the center roll 10, and when it passes through the second part B, it contacts the top roll 15.

Additionally, these felts 21, 22 and 23 can move together with a wet paper, and can absorb water in the wet paper upon pressing the wet paper.

Further, the above-described press part 100 is equipped with doctor blades 10a and 15a contacting press rolls (center roll 10 and top roll 15) of the side contacting the wet paper 30.

Namely, the center roll 10 contacts the doctor blade 10a, and the top roll 15 contacts the doctor blade 15a.

These doctor blades 10a and 15a can remove foreign materials such as materials attached on the surface of the center roll 10 and top roll 15 separated from the wet paper 30.

Further, the press part 100 is equipped with spray nozzles 10b and 15b to apply the functional composition of the present invention on the press rolls (center roll 10 and top roll 15) of the side contacting the wet paper 30.

Namely, the foregoing functional composition of the present invention is used by being applied onto press rolls for dehydration of a wet paper.

Such spray nozzles 10b and 15b are disposed in a downstream side from the places where the doctor blades 10a and 15a are provided to rotation directions R1 and R2 of the center roll 10 and top roll 15.

In this way, by disposing the spray nozzles 10b and 15b in a downstream from the doctor blades 10a and 15a, there is a merit that a detachable membrane is uniformly formed and excess of the functional composition can be removed.

Herein, as the above-described spray nozzles 10b and 15b, there are used an equal sector nozzle, wide sector nozzle, single sector nozzle, empty cone nozzle, filled cone nozzle, filled pyramid nozzle, straight nozzle and the like.

When the wet paper 30 enters to the press part 100, it is pressed at the pressing point P1 via felt 21 by the center roll 10 and first press roll 11 in the first press part A.

By doing so, since water in the wet paper 30 is absorbed by the felt 21, the wet paper 30 is dehydrated.

Next, the wet paper 30 is pressed at the pressing point P2 via felt 22 by the center roll 10 and second press roll 12.

By doing so, since water in the wet paper 30 is absorbed by the felt 22, the wet paper 30 is further dehydrated.

Then, the wet paper 30 is detached from the center roll 10, and transferred to the second press part B. At this time, the foreign material separated from the wet paper 30 is removed by the doctor blade 10a.

The wet paper 30 transferred to the second press part B is pressed at the pressing point P3 via felt 23 by the top roll 15 and third press roll 13.

By doing so, since water in the wet paper 30 is absorbed by the felt 23, the wet paper 30 is further dehydrated.

Then, the wet paper 30 is detached from the top roll 15, and transferred to a dryer part not shown in the figure and dried.

Additionally, the foreign material separated from the wet paper 30 is removed by the doctor blade 15a.

In this way, in the above-described press part 100, by passing a wet paper through between a pair of press rolls via felts, the wet paper is dehydrated.

Further, by applying the foregoing functional composition onto a press roll contacting a wet paper, detachability of the wet paper can be improved.

Further, by applying the foregoing functional composition onto a press roll contacting a doctor blade for removing a foreign material attached onto the surface, abrasion of the doctor blade can be suppressed.

Namely, the above-described functional composition can act as a remover for improving detachability of a wet paper from a press roll, and/or as a friction reducing agent for improving lubricity between the doctor blade and press roll.

As described above, by applying the functional composition onto a press roll, abrasion of a doctor blade can be surely suppressed as well as detachability of a wet paper to the press roll can be surely improved. Therefore, a more stable production becomes possible.

Further, to detach a wet paper from a press roll, in the case where a wet paper is detached away from the surface of press roll by loading a tension in the longitudinal direction of wet paper (hereinafter referred to as “drawing”), a press roll onto which the functional composition of the present invention is applied can loose drawing because detachability of a wet paper to a press roll is improved.

Hence, it is possible to suppress shrinkage of the edge face of a wet paper toward the inside due to excess drawing, and breakage of paper.

Additionally, when the edge face of a wet paper shrinks toward the inside, which gives strain to the paper texture itself, thus when used as a printing paper, color drift takes place.

The preferred embodiments of the present invention have been described so far, but the present invention is not limited to the above-described embodiments.

For example, in the above-described embodiments, when a wet paper 30 is passed through press rolls, one face thereof is contacted with a felt, but both faces of a wet paper may be contacted with felts.

Further, in the present embodiments, of a pair of press rolls, one press roll contacting a wet paper is equipped with a doctor blade, but both the pair of press rolls may be equipped with doctor blades.

### EXAMPLES

Hereinafter, the present invention will be specifically described based on Examples and Comparative examples, and the present invention is not limited to the following Examples.

### Example 1

### Preparation of Functional Composition

In 80% by mass of water, were added 10% by mass of a polyoxyethylene polyoxypropylene block polymer expressed by the following general formula (3) (molecular weight of propylene oxide group: 2000, content ratio of ethylene oxide group: 40% by mass, p=34, q=38, r=38, surface tension: 38.6 mN/m) and 10% by mass of a cationic water-soluble polymer, thereby to obtain a functional composition.

### [STR8]

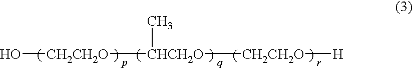

Additionally, the above-described cationic water-soluble polymer is the one that

salt of (meth)acrylic acid with 2-(N,N-dimethylamino)ethylbenzene chloride as a cationic monomer and ethylene glycol mono(meth)acrylate as a nonionic monomer were mixed for the mass ratio to be 1:1, and subjected to a radical polymerization.

### Example 2

A functional composition was obtained in the same manner as in Example 1 except that the content ratio of ethylene oxide group in the polyoxyethylene polyoxypropylene block polymer was set to 80% by mass.

### Example 3

A functional composition was obtained in the same manner as in Example 1 except that the content ratio of ethylene oxide group in the polyoxyethylene polyoxypropylene block polymer was set to 20% by mass.

### Example 4

A functional composition was obtained in the same manner as in Example 1 except that the molecular weight of propylene oxide group in the polyoxyethylene polyoxypropylene block polymer was set to 3000, and the content ratio of ethylene oxide group was set to 80% by mass.

### Example 5

A functional composition was obtained in the same manner as in Example 1 except that the molecular weight of propylene oxide group in the polyoxyethylene polyoxypropylene block polymer was set to 1200.

### Example 6

A functional composition was obtained in the same manner as in Example 1 except that the molecular weight of propylene oxide group in the polyoxyethylene polyoxypropylene block polymer was set to 1500.

### Example 7

A functional composition was obtained in the same manner as in Example 1 except that an amphoteric water-soluble polymer was used in place of the cationic water-soluble polymer.

Additionally, the above-described amphoteric water-soluble polymer is the one that salt of (meth)acrylic acid with 2-(N,N-dimethylamino)ethylbenzene chloride as a cationic monomer, methacrylic acid as an anionic monomer and ethylene glycol mono(meth)acrylate as a nonionic monomer were mixed for the mass ratio to be 5:2:3, and subjected to a radical polymerization.

### Example 8

A functional composition was obtained in the same manner as in Example 1 except that poly(diallyldimethylammonium chloride) was used in place of the cationic water-soluble polymer.

### Example 9

A functional composition was obtained in the same manner as in Example 1 except that dicyandiamide-formamide condensate was used in place of the cationic water-soluble polymer.

### Example 10

A functional composition was obtained in the same manner as in Example 1 except that condensate of epichlorohydrin with dimethylamine was used in place of the cationic water-soluble polymer.

### Example 11

A functional composition was obtained in the same manner as in Example 1 except that the polyoxyethylene polyoxypropylene block polymer was 2%, and the cationic water-soluble polymer was 18% by mass (mass ratio of the polyoxyethylene polyoxypropylene block polymer and water-soluble polymer was 1:9).

### Example 12

A functional composition was obtained in the same manner as in Example 1 except that the polyoxyethylene polyoxypropylene block polymer was 18%, and the cationic water-soluble polymer was 2% by mass (mass ratio of the polyoxyethylene polyoxypropylene block polymer and water-soluble polymer was 1:0.11).

### Example 13

A functional composition was obtained in the same manner as in Example 1 except that the cationic water-soluble polymer was not used.

### Example 14

A functional composition was obtained in the same manner as in Example 1 except that in place of the polyoxyethylene polyoxypropylene block polymer, a compound expressed by the following general formula (1) (hereinafter, simply referred to as “compound A”; content ratio of ethylene oxide group: 55% by mass, carbon numbers of 10 (branched decyl); p+r=6, q=1, surface tension 26.9 mN/m) was used:

### [STR9]

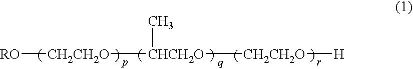

### Example 15

A functional composition was obtained in the same manner as in Example 14 except that in place of compound A, a compound expressed by the above-described general formula (1) (content ratio of ethylene oxide group: 59% by mass, carbon numbers of 10 (branched decyl); p+r=7, q=1, surface tension 27.0 mN/m) was used.

### Example 16

A functional composition was obtained in the same manner as in Example 14 except that in place of compound A, a compound expressed by the above-described general formula (1) (content ratio of ethylene oxide group: 62% by mass, carbon numbers of 10 (branched decyl); p+r=8, q=1, surface tension 27.0 mN/m) was used.

### Example 17

A functional composition was obtained in the same manner as in Example 14 except that in place of compound A, a compound expressed by the above-described general formula (1) (content ratio of ethylene oxide group: 65% by mass, carbon numbers of 10 (branched decyl); p+r=9, q=1, surface tension 27.5 mN/m) was used.

### Example 18

A functional composition was obtained in the same manner as in Example 14 except that in place of compound A, a compound expressed by the above-described general formula (1) (content ratio of ethylene oxide group: 75% by mass, carbon numbers of 10 (branched decyl); p+r=15, q=1, surface tension 27.5 mN/m) was used.

### Example 19

A functional composition was obtained in the same manner as in Example 14 except that in place of compound A, a compound expressed by the above-described general formula (1) (content ratio of ethylene oxide group: 77% by mass, carbon numbers of 10 (branched decyl); p+r=16, q=1, surface tension 27.5 mN/m) was used.

### Example 20

A functional composition was obtained in the same manner as in Example 14 except that in place of compound A, a compound expressed by the above-described general formula (1) (content ratio of ethylene oxide group: 79% by mass, carbon numbers of 10 (branched decyl); p+r=17, q=1) was used.

### Example 21

A functional composition was obtained in the same manner as in Example 14 except that in place of compound A, a compound expressed by the above-described general formula (1) (content ratio of ethylene oxide group: 82% by mass, carbon numbers of 10 (branched decyl); p+r=20, q=1) was used.

### Example 22

A functional composition was obtained in the same manner as in Example 14 except that in place of compound A, a compound expressed by the above-described general formula (1) (content ratio of ethylene oxide group: 82% by mass, carbon numbers of 10 (branched decyl); p+r=21, q=1) was used.

### Example 23

A functional composition was obtained in the same manner as in Example 14 except that in place of compound A, a compound expressed by the above-described general formula (1) (content ratio of ethylene oxide group: 70% by mass, carbon numbers of 9 (linear nonyl); p+r=10, q=1) was used.

### Example 24

A functional composition was obtained in the same manner as in Example 14 except that in place of compound A, a compound expressed by the above-described general formula (1) (content ratio of ethylene oxide group: 67% by mass, mixture of carbon numbers of 10 (branched decyl) and carbon numbers of 12 (linear dodecyl); p+r=10, q=1) was used.

### Example 25

A functional composition was obtained in the same manner as in Example 14 except that in place of compound A, a compound expressed by the above-described general formula (1) (content ratio of ethylene oxide group: 62% by mass, carbon numbers of 12 (linear dodecyl); p+r=7, q=1, surface tension 27.8 mN/m) was used.

### Example 26

A functional composition was obtained in the same manner as in Example 14 except that in place of compound A, a compound expressed by the above-described general formula (1) (content ratio of ethylene oxide group: 66% by mass, carbon numbers of 12 (linear dodecyl); p+r=8, q=0, surface tension 28.5 mN/m) was used.

### Example 27

A functional composition was obtained in the same manner as in Example 14 except that in place of compound A, a compound expressed by the above-described general formula (1) (content ratio of ethylene oxide group: 70% by mass, carbon numbers of 12 (linear dodecyl); p+r=10, q=0, surface tension 31.0 mN/m) was used.

### Example 28

A functional composition was obtained in the same manner as in Example 14 except that in place of compound A, a compound expressed by the above-described general formula (1) (content ratio of ethylene oxide group: 81% by mass, carbon numbers of 12 (linear dodecyl); p+r=18, q=0, surface tension 39.0 mN/m) was used.

### Example 29

A functional composition was obtained in the same manner as in Example 14 except that in place of compound A, a compound expressed by the above-described general formula (1) (content ratio of ethylene oxide group: 64% by mass, carbon numbers of 13 (linear tridecyl); p+r=8, q=0, surface tension 27.5 mN/m) was used.

### Example 30

A functional composition was obtained in the same manner as in Example 14 except that in place of compound A, a compound expressed by the above-described general formula (1) (content ratio of ethylene oxide group: 69% by mass, carbon numbers of 13 (linear tridecyl); p+r=10, q=0, surface tension 27.9 mN/m) was used.

### Example 31

A functional composition was obtained in the same manner as in Example 14 except that in place of compound A, a compound expressed by the above-described general formula (1) (content ratio of ethylene oxide group: 73% by mass, carbon numbers of 13 (linear tridecyl); p+r=12, q=0, surface tension 31.3 mN/m) was used.

### Example 32

A functional composition was obtained in the same manner as in Example 14 except that in place of compound A, a compound expressed by the above-described general formula (1) (content ratio of ethylene oxide group: 82% by mass, carbon numbers of 13 (linear tridecyl); p+r=20, q=0, surface tension 36.3 mN/m) was used.

### Example 33

A functional composition was obtained in the same manner as in Example 14 except that in place of compound A, a compound expressed by the above-described general formula (1) (content ratio of ethylene oxide group: 57% by mass, mixture of carbon numbers of 14 (linear tetradecyl) and carbon numbers of 15 (linear pentadecyl); mixture of p+r=8 and 9, q=1, surface tension 32.0 mN/m) was used.

### Example 34

A functional composition was obtained in the same manner as in Example 14 except that in place of compound A, a compound expressed by the above-described general formula (1) (content ratio of ethylene oxide group: 59% by mass, mixture of carbon numbers of 14 (linear tetradecyl) and carbon numbers of 15 (linear pentadecyl); p+r=9, q=1, surface tension 32.5 mN/m) was used.

### Example 35

A functional composition was obtained in the same manner as in Example 14 except that in place of compound A, a compound expressed by the above-described general formula (1) (content ratio of ethylene oxide group: 64% by mass, mixture of carbon numbers of 14 (linear tetradecyl) and carbon numbers of 15 (linear pentadecyl); p+r=11, q=1, surface tension 34.0 mN/m) was used.

### Example 36

A functional composition was obtained in the same manner as in Example 14 except that in place of compound A, a compound expressed by the above-described general formula (1) (content ratio of ethylene oxide group: 67% by mass, mixture of carbon numbers of 14 (linear tetradecyl) and carbon numbers of 15 (linear pentadecyl); p+r=13, q=1, surface tension 35.0 mN/m) was used.

### Example 37

A functional composition was obtained in the same manner as in Example 14 except that in place of compound A, a compound expressed by the above-described general formula (1) (content ratio of ethylene oxide group: 70% by mass, mixture of carbon numbers of 14 (linear tetradecyl) and carbon numbers of 15 (linear pentadecyl); p+r=15, q=1, surface tension 37.0 mN/m) was used.

### Example 38

A functional composition was obtained in the same manner as in Example 14 except that in place of compound A, a compound expressed by the above-described general formula (1) (content ratio of ethylene oxide group: 61% by mass, carbon numbers of 16 (linear hexadecyl); p+r=10, q=1) was used.

### Example 39

A functional composition was obtained in the same manner as in Example 14 except that in place of compound A, a compound expressed by the above-described general formula (1) (content ratio of ethylene oxide group: 60% by mass, carbon numbers of 16 (linear hexadecyl) and carbon numbers of 18 (linear octadecyl); p+r=10, q=1) was used.

### Example 40

A functional composition was obtained in the same manner as in Example 36 except that an amphoteric water-soluble polymer was used in place of the cationic water-soluble polymer.

Additionally, the above-described amphoteric water-soluble polymer is the one that salt of (meth)acrylic acid with 2-(N,N-dimethylamino)ethylbenzene chloride as a cationic monomer, methacrylic acid as an anionic monomer and ethylene glycol mono(meth)acrylate as a nonionic monomer were mixed for the mass ratio to be 5:2:3, and subjected to a radical polymerization.

### Comparative Example 1

A functional composition was obtained in the same manner as in Example 1 except that alkyldimethylbenzalkonium chloride was used in place of the polyoxyethylene polyoxypropylene block polymer.

### Comparative Example 2

A functional composition was obtained in the same manner as in Example 1 except that only water-soluble polymer was used without using the polyoxyethylene polyoxypropylene block polymer.

(Evaluation Method)

In a press part of a paper making machine shown in FIG. 1, the functional compositions obtained in the above-described Examples 1 to 40 and Comparative examples 1, 2 were each applied onto a center roll (press roll), detachability of a wet paper and abrasion of a doctor blade were examined.

Additionally, operating conditions of actual equipment used in test are as follows:

Paper category: coated paper
Base weight: 44 g/m²
Paper width: 5 m
Rotation speed of center roll: 1100 m/min
Material of center roll: ceramic-sprayed roll
Material of doctor blade: carbon
Linear pressure of doctor blade: 350 g/cm
Rotation speed of top roll: 1140 m/min
Material of top roll: ceramic-sprayed roll
Applied amount of functional composition: 25 cc/min
Applied amount of water to center roll: 50 L/min

[Detachability Based on Actual Equipment]

FIG. 2 is an explanatory diagram for explaining detachability test based on actual equipment.

As shown in FIG. 2, in a state that no functional composition is applied, when a press roll is operated at a predetermined speed S1, a wet paper 30 is detached at a detaching point T1.

On the other hand, in a state that each functional composition was applied, when a press roll is operated at the same speed S1, a wet paper 30 is detached at a detaching point T2.

Additionally, since the detaching point is moving up and down in operation, the position at a center of the movement is defined as a detaching point.

Subsequently, operation speed is reduced in a state that each functional composition was applied.

By doing so, a force in drawing becomes weak, the detaching point T2 is gradually moving toward the direction of the detaching point T1.

Then, a speed S2 when the detaching point T2 consists with the detaching point T1 is measured.

The difference between the speeds S1 and S2 was defined as degree of detachability.

The result obtained is shown in Table 1.

[Abrasion Property]

Further, in a state that each functional composition was applied, the abrasion amount of a doctor blade when actual equipment was operated for 2 weeks was measured in terms of per one day.

The result obtained is shown in Table 1.

Additionally, abrasion property in each Example is shown as a normalized value (relative value to blank value) provided that abrasion amount is 100 when only water is applied (blank test).

[Detachability of Wet Paper]

Next, as an adjunctive test to show the effect of the functional composition of the present invention, detachability test of wet paper was conducted.

The method will be explained below by using figures.

FIGS. 3 (A) and (B) are explanatory diagrams for explaining the detachability test of wet paper.

As shown in FIG. 3, 5 cc of each functional composition diluted with water by 2000 times was applied onto the entire face of the upper surface of ceramic-sprayed plate 51.

After that, a wet paper 30 was placed on the ceramic-sprayed plate 51, and a felt 52 is placed on the wet paper 30 to be a laminated body.

Then, this laminated body was pressed by press rolls 53 made of metal and the laminated body was transferred in a horizontal direction so that the entirety of wet paper 30 is pressed under an uniform pressure.

In this way, the wet paper 30 was dehydrated.

Additionally, the ratio of the weight of the part that water was removed from the wet paper to the weight of wet paper in the wet paper 30 (dryness) was about 38%.

After that, a hook with a wire 54 was attached to a terminal part of wet paper 30, this hook with a wire 54 was hooked to a pulley capable of moving horizontally, and the distal end of the wire was connected to a load cell 55 (manufactured by Kyowa Electronic Instrument Co., Ltd.).

Then, the pulley was moved to detach the wet paper 30 from the ceramic-sprayed plate 51 while keeping a constant angel (θ=15°) when a wet paper leaves the actual equipment actually.

At that time, detachment force shown by the load cell 55 was measured.

The result obtained is shown in Table 1.

Additionally, abrasion force in each Example is shown as a normalized value (relative value to blank value) provided that abrasion force is 100 when only water is applied onto the ceramic-sprayed plate 51 (blank test).

[Dynamic Friction Force Measurement]

Next, a test was carried out to make sure the effect that the functional composition of the present invention lowers dynamic friction force.

FIG. 4 is an explanatory diagram for explaining dynamic friction force measurement test.

As shown in FIG. 4, 5 cc of the functional composition diluted with water by 2000 times was applied onto the entire face of ceramic-sprayed plate 51.

After that, a doctor blade 56 made of carbon standing at a predetermined angle (α=30°) to a ceramic-sprayed plate 51 was connected to a load cell 55 with a wire, and the load cell 55 was connected to a motor 57 with a wire.

Then, the load cell 55 was pulled by the motor 57, dynamic friction force shown by the load cell 55 was measured during the doctor blade 56 made of carbon slid the ceramic-sprayed plate 51.

The result obtained is shown in Table 1.

Additionally, dynamic friction force in each Example is shown as a normalized value (relative value to blank value) provided that dynamic friction force is 100 when only water is applied onto the ceramic-sprayed plate 51.

TABLE 1

| | Detachability | | | |
|---|---|---|---|---|
| | based on actual | | | Dynamic |
| | equipment | Abrasion | Detachability | friction force |
| | (m/min) | property | of wet paper | measurement |
| Example 1 | 38 | 59 | 45 | 50 |
| Example 2 | 37.7 | 47 | 43 | 45 |
| Example 3 | 38.5 | 70 | 65 | 54 |
| Example 4 | 37.5 | 55 | 40 | 40 |
| Example 5 | 38.8 | 85 | 65 | 66 |
| Example 6 | 38.5 | 68 | 65 | 62 |
| Example 7 | 38 | 57 | 45 | 48 |
| Example 8 | 38.2 | 60 | 47 | 50 |
| Example 9 | 39.5 | 59 | 45 | 49 |
| Example 10 | 39 | 57 | 47 | 45 |
| Example 11 | 39.5 | 80 | 60 | 65 |
| Example 12 | 39.8 | 65 | 45 | 58 |
| Example 13 | 39.5 | 75 | 50 | 50 |
| Example 14 | 39.2 | 70 | 60 | 58 |
| Example 15 | 39.1 | 65 | 57 | 55 |
| Example 16 | 39 | 66 | 56 | 56 |
| Example 17 | 38.6 | 55 | 53 | 53 |
| Example 18 | 38.5 | 56 | 50 | 51 |
| Example 19 | 38.3 | 56 | 54 | 51 |
| Example 20 | 38.8 | 59 | 56 | 57 |
| Example 21 | 39.1 | 60 | 56 | 58 |
| Example 22 | 39.8 | 64 | 60 | 61 |
| Example 23 | 39.8 | 65 | 66 | 68 |
| Example 24 | 38.2 | 55 | 54 | 51 |
| Example 25 | 38.7 | 60 | 55 | 55 |
| Example 26 | 38.4 | 58 | 53 | 50 |
| Example 27 | 37.4 | 48 | 40 | 38 |
| Example 28 | 38 | 54 | 50 | 53 |
| Example 29 | 38.2 | 54 | 47 | 52 |
| Example 30 | 37.0 | 46 | 42 | 44 |
| Example 31 | 37.0 | 47 | 40 | 40 |
| Example 32 | 38 | 51 | 51 | 54 |
| Example 33 | 37.8 | 50 | 48 | 47 |
| Example 34 | 37.2 | 48 | 40 | 40 |
| Example 35 | 37.2 | 50 | 40 | 44 |
| Example 36 | 37.1 | 48 | 41 | 43 |
| Example 37 | 37.8 | 50 | 43 | 46 |
| Example 38 | 38.3 | 55 | 48 | 50 |
| Example 39 | 39.4 | 60 | 66 | 70 |
| Example 40 | 37.3 | 52 | 40 | 39 |
| Comparative | 40.3 | 96 | 102 | 97 |
| example 1 | | | | |
| Comparative | 39.8 | 95 | 90 | 95 |
| example 2 | | | | |
| Water | 40 | 100 | 100 | 100 |
| (blank test) | | | | |

As is clear from the result shown in Table 1, according to the functional compositions of Examples 1 to 40, it has been known that detachability based on actual equipment, abrasion property, detachability of wet paper and dynamic friction force are all excellent compared with the functional compositions of Comparative examples 1 and 2.

From this, according to the present invention, it has been confirmed to provide a functional composition capable of improving detachability of a wet paper to a press roll by being applied onto a press roll, and enables a stable paper production because abrasion of a doctor blade can be suppressed, and a method for improvement in detachability of a wet paper using the functional composition.

### INDUSTRIAL APPLICABILITY

The functional composition of the present invention is used in a paper making machine comprising felt, a pair of press rolls for pressing a wet paper via the felt, and a doctor blade contacting a press roll of the side contacting a wet paper, a stable production becomes possible because by being applied onto this press roll, abrasion of the doctor blade can be suppressed as well as detachability of the wet paper to the press roll can be improved. Description of symbol and number

### BRIEF DESCRIPTION OF THE DRAWINGS

FIG. 1 is an explanatory diagram for explaining one example of constitution of a press part in a paper making machine that the functional composition of the present invention is used.

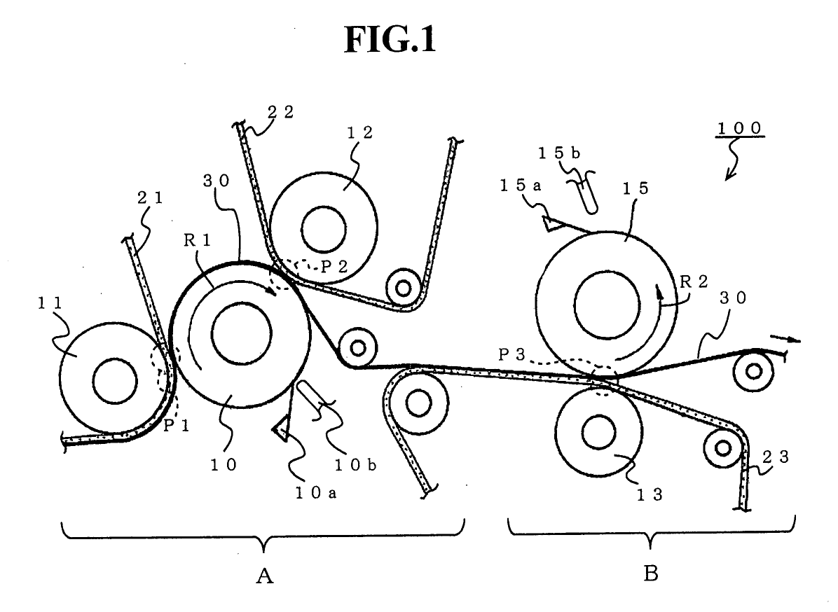

FIG. 2 is an explanatory diagram for explaining detachability test based on actual equipment in Examples.

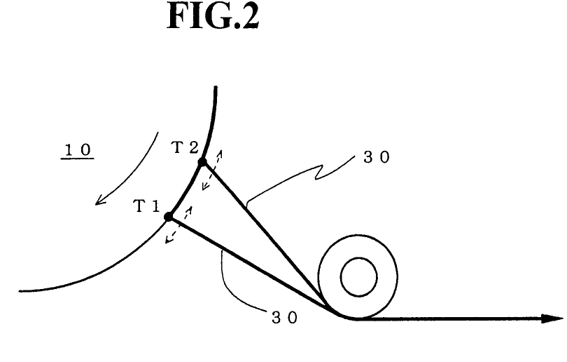

FIGS. 3 (A) and (B) are explanatory diagrams for explaining detachability test of wet paper in Examples.

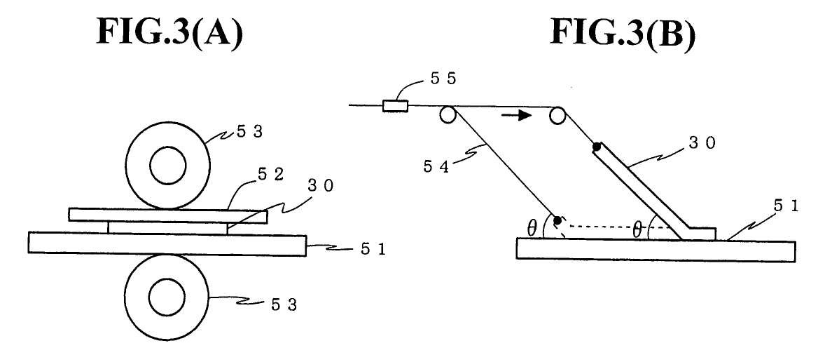

FIG. 4 is an explanatory diagram for explaining dynamic friction force measurement test.

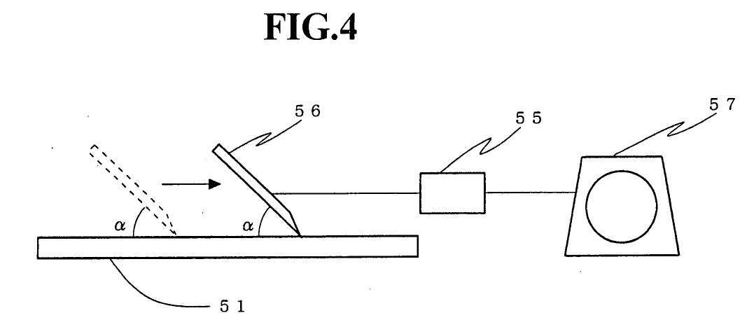

FIG. 5 is an explanatory diagram for explaining detachability of wet paper on press rolls.

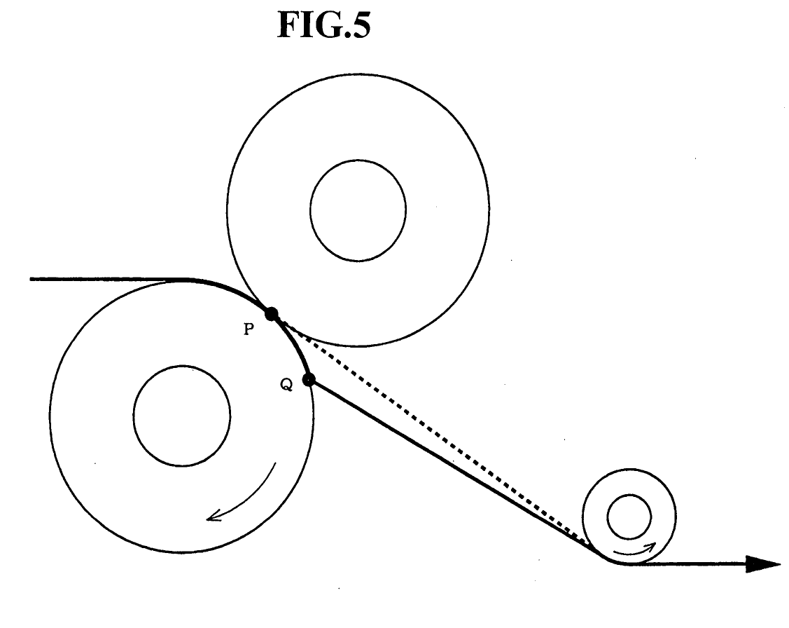

| | |
|---|---|
| 10 | Center roll (press roll) |
| 10a, 15a, 56 | Doctor blade |
| 10b, 15b | Spray nozzle |
| 11, 12, 13, 53 | Press roll |
| 15 | Top roll (press roll) |
| 21, 22, 23, 52 | Felt |
| 30 | Wet paper |
| 51 | Ceramic-sprayed plate |
| 54 | Hook with wire |
| 55 | Load cell |
| 57 | Motor |
| 100 | Press part |
| A | First press part |
| B | Second press part |
| P, P1, P2, P3 | Pressing point |
| Q | Point |
| R1, R2 | Rotation direction |
| T1, T2 | Detaching point |

1. A functional composition used by being applied onto a press roll for dehydration of a wet paper, comprising a compound expressed by the following general formula (1):
[STR1] 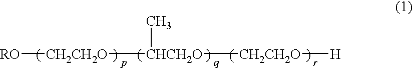
wherein R represents an organic group which may have a substituent or a hydrogen atom, p and r each independently represent an integer of 0 to 228, and q represents an integer of 0 to 69, provided that p, q and r do not represent 0 simultaneously.

2. The functional composition according to claim 1, further comprising a water-soluble polymer.

3. The functional composition according to claim 1, wherein said R is a hydrogen atom, said p and r each independently are an integer of 27 to 228, said q is an integer of 25 to 69.

4. The functional composition according to claim 2, wherein said R is a hydrocarbon group having carbon numbers of 10 to 16 which may have a substituent, the sum of said p and r is an integer of 6 to 30, and said q is an integer of 0 to 2.

5. The functional composition according to claim 1, wherein the content ratio of ethylene oxide group expressed by the following general formula (2) to the molecular weight of the compound expressed by the foregoing general formula (1) is 30% by mass or more.
[STR2]
CH₂CH₂O (2)

6. The functional composition according to claim 2, wherein the mixing ratio of the compound expressed by the foregoing general formula (1) and said water-soluble polymer is 1:0.1 to 1:10 in mass ratio.

7. The functional composition according to claim 1, wherein said press roll includes a doctor blade for removing a foreign material attached on the surface.

8. The functional composition according to claim 1, which is a remover to improve detachability of said wet paper from said press roll.

9. The functional composition according to claim 7, which is a friction reducing agent to improve lubricity between said doctor blade and said press roll.

10. A method for improvement in detachability of a wet paper using the functional composition of claim 1.
